(19) United States
(12) Patent Application Publication
(10) Pub. No.: US 2007/0128191 A1
(43) Pub. Date: Jun. 7, 2007

(54) Polyclonal antibodies, preparation method thereof and use of same

(75) Inventor: Manuel Sarasa Barrio, Zaragoza (ES)

Correspondence Address: CROWELL & MORING LLP;INTELLECTUAL PROPERTY GROUP, P.O. BOX 14300, WASHINGTON, DC, 20044-4300, US

(73) Assignee: UNiversidad De Zaragoza, Zaragoza (ES)

(21) Appl. No.: 10/527,742

(22) PCT Filed: Aug. 13, 2003
(86) PCT No.: PCT/ES03/00422

§ 371 Date: Nov. 16, 2005

(30) Foreign Application Priority Data
Sep. 12, 2002 (ES) P 0202094

(51) Int. Cl. A61K 39/395; C07K 16/28

(52) U.S. Cl. 424/145.1; 530/389.1; 530/391.1

(57) ABSTRACT

Antibodies that specifically bind to amyloid beta peptide Aβ40 or Aβ42, obtainable by immunization of a mammal with a polypeptide conjugated to a peptide comprising an amino acid sequence selected from the group consisting of SEQ ID NOs: 1, 2, 3, and 4, and method for preparing the antibodies. Preferably, the polypeptide is a conjugate of the peptide and keyhole limpet hemocyanin (KLH), and the mammal is a rabbit. Also provided are isolated polypeptides comprising an amino acid sequence of SEQ ID NO: 1, 2, 3 or 4, Further provided is a method of detecting the presence or absence of amyloid peptide Aβ40 or Aβ42 in a specimen, and a method of evaluating the ability of a substance in activating the degradation of the amyloid peptide or in inhibiting their production.

The present invention relates to polyclonal antibodies which recognize, specifically and with great affinity for, the two most important amyloid peptides, Aβ40 and Aβ42, as well as their use in evaluating both drugs activating the degradation of the amyloid peptides characteristic of Alzheimer's disease and drugs inhibiting their formation. In the same way, they can be useful for evaluating the activity of the enzymes involved in the processing of the precursor protein of the two amyloid peptides or the activity of the enzymes involved in the degradation of same, as well as for evaluating the level of expression of the genes involved in the entire chain of events which lead to the deposition and formation of amyloid plaques, lesions characteristic of the brains of patients suffering from Alzheimer's disease.

### BACKGROUND OF THE INVENTION

Certain factors are known about the biochemical and metabolic phenomena associated with the presence of Alzheimer's disease. Two morphological and histopathological changes observed in the brains of patients with Alzheimer's disease are neurofibrillar tangles (MNF) and amyloid deposits.

Intraneuronal neurofibrillar tangles are also present in other degenerative diseases but the presence of amyloid deposits both in the intemeuronal spaces (neuritic plaques) and in the surrounding microvasculature (vascular plaques) seems to be characteristic of Alzheimer's disease. Of these, the neuritic plaques seem to be the most characteristic (Price, D. L. et al., Drug

The main component of these amyloid plaques is a peptide of 40-42 amino acids called amyloid peptide Aβ4.

Amyloid peptide Aβ4 is a polypeptide produced by proteolysis from membrane glucoproteins called amyloid peptide Aβ4 precursor proteins (BAPP). These amyloid peptide precursor proteins are made up of 695 to 770 amino acids, and are all encoded by the same gene.

Two main variants of amyloid peptide Aβ4, peptide Aβ40 and Aβ42, with 40 and 42 amino acids respectively, have been identified which present a different tissue distribution both in physiological and in pathological conditions.

We have cloned and sequenced the BAPP gene in the chicken and have shown that it is practically identical to the human gene since it produces BAPPs which are highly homologous, in the order of 95%, with those of the human species, and the AB4 peptide characteristic of Alzheimer's disease is identical to the human one. Furthermore, the chicken embyro processes βAPPs in such a way that peptide Aβ4 is produced, due to the action of proteolytic enzymes which cause the proteolysis of the βAPPs in a key site to produce Aβ4; the proteolytic enzyme which cuts βAPPs to produce Aβ4 is called β-secretase.

### DESCRIPTION OF THE INVENTION

The present invention provides polyclonal antibodies capable of specifically recognizing by means of any conventional immunological technique (western blot, immunohistochemistry, immunoprecipitation, ELISA, RIA, etc.) the presence of the amyloid peptides Aβ40 and Aβ42. The antibodies are obtained by immunization of mammals, preferably rabbits, with a protein conjugated with a peptide selected from a group consisting of SEQ ID NO: 1, SEQ ID NO: 2, SEQ ID NO: 3, SEQ ID NO: 4, optionally shorthened by elimination of the amino acid radicals of the N-terminal and/or C-terminal ends, and optionally lengthened by adding the appropriate amino acid radicals to conjugate the protein.

In a particular embodiment, the peptide corresponds to SEQ ID NO: 1, optionally lengthened by adding the appropriate amino acid radicals to conjugate the protein. In another particular embodiment, the peptide corresponds to SEQ ID NO 2:, optionally lengthened by adding the appropriate amino acid radicals to conjugate the protein. In another particular embodiment, the peptide corresponds to SEQ ID NO 3, optionally lengthened by adding the appropriate amino acid radicals to conjugate the protein. In another particular embodiment, the peptide corresponds to SEQ ID NO 4, optionally lengthened by adding the appropriate amino acid radicals to conjugate the protein. Though the elimination of the terminal amino acid radicals does not eliminate the specific activity, the preferred peptides are those of SEQ ID NO 1, SEQ ID NO 2, SEQ ID NO 3 and SEQ ID NO 4.

The provision of any of the substantially pure peptides mentioned above is also part of the present invention.

This invention also provides a method for obtaining the polycloncal antibodies mentioned above by immunization of mammals, preferably rabbits, with a protein conjugated to a peptide selected from a group consisting of SEQ ID NO 1, SEQ ID NO 2, SEQ ID NO 3 and SEQ ID NO 4 optionally shortened by elimination of the amino acid radicals of the N-terminal and/or C-terminal ends, and optionally lengthened by adding the appropriate amino acid radicals to conjugate the protein.

According to a preferred embodiment of the present invention, the protein used for its conjugation with the peptide is keyhole limpet hemocyanin.

In an even more preferred embodiment of the present invention, the mammals used for their immunization with the protein conjugated to the peptide are rabbits.

According to an aspect of the present invention, a new method is provided for the evaluation both of drugs activating the degradation of the amyloid peptides characteristic of Alzheimer's disease and drugs inhibiting their production by means of the use of the polyclonal antibodies described above.

Similarly, the method also serves to evaluate the activity of the enzymes (proteases) involved in the processing of the precursor protein of the peptides cited or the activity of the enzymes involved in the degradation of same.

This invention also provides a method for the detection of the presence or absence of the amyloid peptides Aβ40 and Aβ42 in a specimen, using the chicken embryo or any of the extraembryonic membranes or fluids of the embryonated chicken egg as an animal test model.

According to a preferred embodiment of the present invention, a new method is provided for the evaluation both of drugs activating the degradation of the amyloid peptides characteristic of Alzheimer's disease and drugs inhibiting their production by means of the use of the chicken embryo or any of the extraembryonic membranes or fluids of the embryonated chicken egg as an animal test model.

According to another preferred embodiment of the present invention, a new method is provided for the evaluation of the activity of the enzymes (proteases) involved in the processing of the precursor protein of the peptides cited or the activity of the enzymes involved in the degradation of same by means of the use of the chicken embryo or any of the extraembryonic membranes or fluids of the embryonated chicken egg as an animal test model.

The method comprises of inoculating the drug into the embryonated chicken egg whether by simply dropping it onto the embryo itself or any of its membranes or by injecting it into the vitellus (if the embryo is young) or the vitelline sac (if the embryo is bigger), into the amniotic sac, into the allantoid sac (in embryos incubated for more than 6 days) or in the inside of the embryo itself, after adequate incubation time, the embryo and/or any of the extraembryonic membranes or fluids are extracted and the quantity of amyloid peptides characteristic of Alzheimer's disease is analyzed by means of conventional laboratory techniques for the quantification of peptides and proteins such as western blot, immunohistochemistry, immunoprecipitation, ELISA, RIA, HPLC, etc.

### EXAMPLES

The present invention is illustrated by the following examples.

### Example 1

### Coupling the Peptides to Keyhole Limpet Hemocyanin (KLH)

The peptides were coupled to keyhole limpet hemocyanin via the n-terminus using the coupling agent glutaraldehyde. For this purpose the KLH protein was activated in a pH 10 borate buffer solution. The synthetic peptide was then added and the 0.3% glutaraldehyde solution was slowly added with stirring at ambient temperature. After the addition of glycine 1M to block the non-reacting glutaraldehyde, the peptide-protein conjugate was dialyzed against 3 liters of pH 8.5 borate buffer at a temperature of 4° C. The peptide-KLH conjugate was stored at 4° C.

### Example 2

### Generation of Polyclonal Antibodies

The four polyclonal antibodies were generated by immunization of New Zealand White rabbits against the four peptides coupled to KLH which are used as an immunogen.

Each immunogen was injected into two rabbits, with five injections being performed: the first intradermic injection of the peptide-KLH conjugate in PBS and emulsified in complete Freund's adjunct and four other intramuscular ones by way of a booster dose on days 14, 28, 49 and 80 of the same peptide-KLH conjugate in PBS but this time emulsified in incomplete Freund's adjunct, with the blood sampling being performed at 90 days to detect the presence of antibodies.

### Example 3

### Purification of the Antibodies by Affinity

After the blood was drawn, the serum was separated and prepurified by means of desalting and the antibodies were then purified by affinity in a matrix composed of 1.5 ml of EMD epoxy-activated material (Merck) to which 5 mg of the corresponding peptide were added. The purified fractions were established in 0.1% BSA (Sigma) and stored at 4° C., with glycerol 20-50% possibly being added as a cryoprotectant.

### Example 4

### Antibody Titration by ELISA

After purification by affinity, the antibody titer was determined by ELISA. For this, the antigen was placed in an ELISA Maxi Sorb plate from Nunc at a rate of 50 ng/50 μl in pH 7 PBS and the antibody was detected with donkey anti-IgG conjugated with alkaline phosphatase, using p-nitrophenyl phosphate (PNPP) in diethanolamine with 5 mM MgCl₂, pH 9.6, as a substrate and developed at 2 hours.

In conclusion, the antibodies were generated using the different synthetic peptides described above coupled with KLH. These synthetic peptides contain a very small number of amino acids, which makes them highly suitable for the chain production of homogeneous antibodies with predefined epitopes

LIST OF SEQUENCES

| | |
|---|---|
| SEQ ID NO 1 | LVFFAEDV |
| SEQ ID NO 2 | GLMVGGVV |
| SEQ ID NO 3 | GLMVGGVVIA |
| SEQ ID NO 4 | RHDSGYEVHHQK |

In this application the amino acids are abbreviated using the one-letter codes accepted in the field, in the form shown below:
A=Ala=alanine
C=Cys=cysteine
D=Asp=aspartic acid
E=Glu=glutamic acid
F=Phe=phenylalanine
G=Gly glycine
H=His=histidine
I=lie=isoleucine
K=Lys=lysine
L=Leu=leucine
M=Met=methionine
N=Asn=asparagine
P=Pro=proline
Q=Gln=glutamine
R=Arg=arginine
S=Ser=serine
T=Thr=threonine
V=Val=valine
W=Trp=tryptophan
Y=Tyr=tyrosine

The information relating to the identification of the peptide sequences described in the present invention which accompanies the present record in a form readable by computer is identical to the listing of sequences presented with the record.

NUMBER OF SEQUENCES: 4
INFORMATION ON SEQUENCE 1:
CHARACTERISTICS OF THE SEQUENCE:
LONGITUDE: 8
TYPE: amino acid
TYPE OF MOLECULE: peptide
SOURCE: Chemical Synthesis
DESCRIPTION OF THE SEQUENCE:
SEQ ID NO 1
Leu Val Phe Phe Ala Glu Asp Val
1 5
INFORMATION ON SEQUENCE 2:
CHARACTERISTICS OF THE SEQUENCE:
LONGITUDE: 8
TYPE: amino acid
TYPE OF MOLECULE: peptide
SOURCE: Chemical Synthesis
DESCRIPTION OF THE SEQUENCE:
SEQ ID NO 2
Gly Leu Met Val Gly Gly Val Val
1 5
INFORMATION ON SEQUENCE 3:
CHARACTERISTICS OF THE SEQUENCE:
LONGITUDE: 10
TYPE: amino acid
TYPE OF MOLECULE: peptide
SOURCE: Chemical Synthesis
DESCRIPTION OF THE SEQUENCE:
SEQ ID NO 3
Gly Leu Met Val Gly Gly Val Val Ile Ala
1 5 10
INFORMATION ON SEQUENCE 4:
CHARACTERISTICS OF THE SEQUENCE:
LONGITUDE: 12
TYPE: amino acid
TYPE OF MOLECULE: peptide
SOURCE: Chemical Synthesis
DESCRIPTION OF THE SEQUENCE:
SEQ ID NO 4
Arg His Asp Ser Gly Tyr Glu Val His His Gln Lys
1 5 10

1-26. (canceled)

27. An antibody that specifically binds to amyloid beta peptide Aβ40 or Aβ42, obtainable by immunization of a mammal with a polypeptide conjugated to a peptide comprising an amino acid sequence selected from the group consisting of SEQ ID NOs: 1, 2, 3, and 4; or to a peptide with a sequence resulting from eliminating one or more N-terminal or C-terminal amino acid residues of SEQ ID NOs:1, 2, 3, or 4; or to a peptide with a sequence resulting from adding one or more N-terminal or C-terminal amino acid residues of SEQ ID NOs:1, 2, 3, or 4.

28. The antibody according to claim 27, wherein the immunization is performed with a peptide selected from the group consisting of a polypeptide conjugated to a peptide comprising an amino acid sequence selected from the group consisting of SEQ ID NOs: 1, 2, 3, and 4.

29. The antibody according to claim 28, wherein the immunization is performed with a peptide selected from the group consisting of a polypeptide conjugated to a peptide consisting of an amino acid sequence selected from the group consisting of SEQ ID NOs: 1, 2, 3, and 4.

30. The antibody according to claim 27, wherein the polypeptide is a conjugate of the peptide and keyhole limpet hemocyanin (KLH).

31. The antibody according to claim 27, wherein the mammal is a rabbit.

32. An isolated polypeptide comprising an amino acid sequence of SEQ ID NO: 1, 2, 3 or 4, or an amino acid sequence of SEQ ID NO: 1, 2, 3, or 4.

33. A method for preparing an antibody, comprising conjugating the isolated polypeptide of claim 32 to form a polypeptide, and immunizing a mammal with the polypeptide.

34. The method according to claim 33, wherein the peptide is conjugated to keyhole limpet hemocyanin (KLH).

35. The method according to claim 34, wherein the mammal is a rabbit.

36. The method according to claim 33, wherein the antibody specifically recognizes amyloid beta peptide Aβ40 or Aβ342.

37. A method of detecting the presence or absence of amyloid peptide Aβ40 or Aβ42 in a specimen, comprising placing said specimen in contact with an antibody according to claim 27, and detecting the presence or absence of a complex formed by said amyloid peptide and said antibody.

38. A method of evaluating the ability of a substance in activating the degradation of the amyloid peptide or in inhibiting their production, the method comprising introducing an antibody according to claim 27 to an embryonated chicken egg.

39. The method according to claim 38, further comprising determining the presence of an complex formed between the antibody and the amyloid peptide.


SEQUENCE LISTING (4 sequences)

SEQ ID NO: 1 - protein, 8 aa, Artificial Sequence
  Description of Artificial Sequence Synthetic Peptide
LVFFAEDV

SEQ ID NO: 2 - protein, 8 aa, Artificial Sequence
  Description of Artificial Sequence Synthetic Peptide
GLMVGGVV

SEQ ID NO: 3 - protein, 10 aa, Artificial Sequence
  Description of Artificial Sequence Synthetic Peptide
GLMVGGVVIA

SEQ ID NO: 4 - protein, 12 aa, Artificial Sequence
  Description of Artificial Sequence Synthetic Peptide
RHDSGYEVHHQK